(19) United States
(12) Patent Application Publication
(10) Pub. No.: US 2016/0095753 A1
(43) Pub. Date: Apr. 7, 2016

(54) Method of Using Absorbent Device for Cleaning and Drying Purposes

(71) Applicant: Teddy Garcia, Orange Park, FL (US)

(72) Inventor: Teddy Garcia, Orange Park, FL (US)

(21) Appl. No.: 14/877,067

(22) Filed: Oct. 7, 2015

Related U.S. Application Data
(60) Provisional application No. 62/060,848, filed on Oct. 7, 2014.

(51) Int. Cl. A61F 11/00; A61B 17/24

(52) U.S. Cl. CPC A61F 11/006; A61B 17/24; A61B 2017/0023

(57) ABSTRACT

A method of cleaning and drying a hollow cavity with a cleaning apparatus which is made of a flexible, soft, and moisture absorbent material. A structural body of the cleaning apparatus contains a first wide base, a first narrow base, and a first conical lateral surface. The conical shape of the structural body allows the user to maintain a firm grasp during the cleaning and drying process. The cleaning process can be completed by either scarping an inner surface of the hollow cavity or by dabbing the inner surface of the hollow cavity. The flexible and soft material ensures that no damage is caused to the inner surface during the cleaning and drying process.

The current application claims a priority to the U.S. Provisional Patent application Ser. No. 62/060,848 filed on Oct. 7, 2014.

### FIELD OF THE INVENTION

The present invention relates generally to a method of using an ear cleaning and drying apparatus for cleaning ear wax. More specifically, the apparatus utilized in the present invention ensures that the ear drum of the user is not damaged during the cleaning and drying process.

### BACKGROUND OF THE INVENTION

Many people will resort to cotton swabs or a simple “do-it-yourself” solution for cleaning and/or drying their ears. Even though cotton swabs and other comparable cleaning methods have certain advantages, they have their drawbacks too. As an example, cotton swabs are designed as a small stick having a bulb-shaped cotton wad wrapped around one or both ends. Therefore, if the cotton swabs are improperly used for ear cleaning, the cotton swabs can result in trauma to the ear canal and potentially rupture the tympanic membrane. As an example, the relative rigidity and the coarse texture of the cotton swab can cause abrasion or bleeding within the ear canal.

Another disadvantage with cotton swabs is the inability to fully absorb moisture within the ear canal. As a result of the low absorbent qualities, existing cotton swabs cannot be used for drying the ear canal. Therefore, the need for a device that has the ability to clean and dry the ear canal simultaneously is clearly evident.

As discussed earlier, cotton swabs are rigid in structure. As a result, when the cotton swab is within the ear canal, the cotton swab does not deform according to the shape of the ear canal. The inability to change according to the shape of the ear canal has certain disadvantages. As an example, the user is unable to judge the depth the cotton swab has travelled within the ear canal. Therefore, the probability of the rigid cotton swab damaging the delicate tympanic membrane, which is easily ruptured, is relatively high. The high probability of damaging the tympanic membrane has led to many medical experts advising against the use of cotton swabs or other comparable “do-it-yourself” methods.

In order to prevent undue amount of wax build up inside the ear canal, which can result in infections and/or the loss of hearing, the ear canal does need to be occasionally cleaned of wax build-up. A trip to a medical professional may not always be practical or convenient. Therefore, there is a need for a tool which a common individual may use to clean his or her ears safely and effectively. The present invention addresses the aforementioned issues and drawbacks of the current “do-it-yourself” ear cleaning methods by providing a simple yet effective and safe way to dry and clean the ear canal. The device introduced by the present invention allows the user to have more control when cleaning the ear canal. As a result, any damages that can occur within the ear canal and the tympanic membrane are prevented.

### BRIEF DESCRIPTION OF THE DRAWINGS

FIG. 1A is a flowchart illustrating the basic overall method of the present invention; and

FIG. 1B is a flowchart thereof, further illustrating the basic overall method of the present invention.

FIG. 2 is a flowchart illustrating the process of scraping during the cleaning process.

FIG. 3 is a flowchart illustrating the process of dabbing during the cleaning process.

FIG. 4 a flowchart illustrating the process of using the cleaning apparatus in an ear canal.

FIG. 5 a flowchart illustrating the process of using the cleaning apparatus in a nasal cavity.

FIG. 6 is a perspective view of the first structural body of the cleaning apparatus.

FIG. 7 is a bottom perspective view of the first structural body, wherein a solid core area is represented.

FIG. 8 is a bottom perspective view of the first structural body, wherein a hollow core area is represented.

FIG. 9 is a perspective view of the cleaning apparatus, wherein the first structural body and the second structural body are illustrated.

FIG. 10 is an illustration of aligning the cleaning apparatus with the hollow cavity.

FIG. 11 is a flowchart illustrating the basic overall method of the present invention, wherein the second structural body is used.

FIG. 12 is a flowchart illustrating the process of utilizing the second structural body for scraping during the cleaning process.

FIG. 13 is a flowchart illustrating the process of utilizing the second structural body for dabbing during the cleaning process.

FIG. 14 is a flowchart illustrating the process of using the cleaning apparatus in a second ear canal.

FIG. 15 is a flowchart illustrating the process of using the cleaning apparatus in a second nasal cavity.

FIG. 16 is another illustration of the cleaning apparatus.

### DETAIL DESCRIPTIONS OF THE INVENTION

All illustrations of the drawings are for the purpose of describing selected versions of the present invention and are not intended to limit the scope of the present invention.

The present invention introduces a method of cleaning and drying an ear canal or a nasal cavity with a cleaning apparatus. The cleaning apparatus is specifically designed to be user-friendly and cause no damage to the inner surface of the nasal cavity or the inner surface of the ear canal. By following the method introduced in the present invention, the user is guaranteed to avoid any harmful side effects that can occur from cotton swabs and other comparable equipment that are currently used.

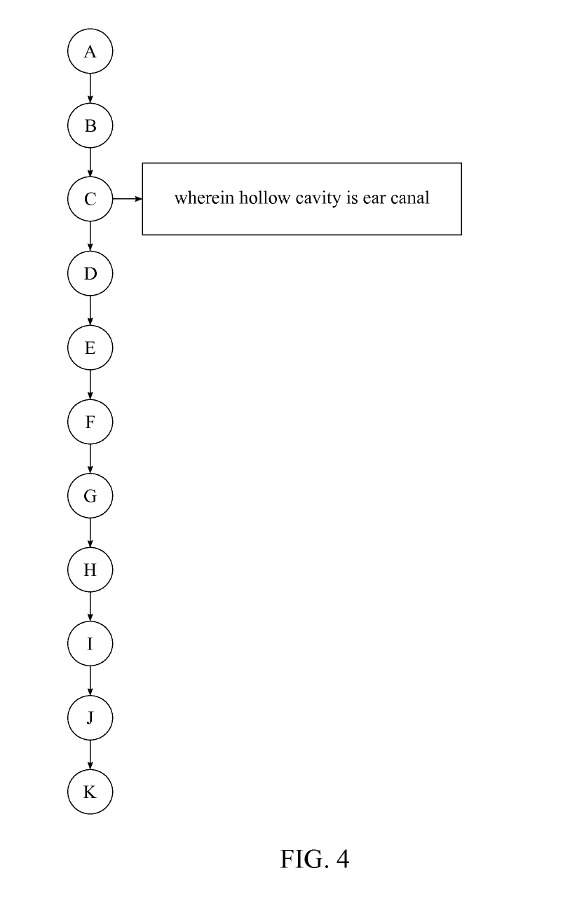

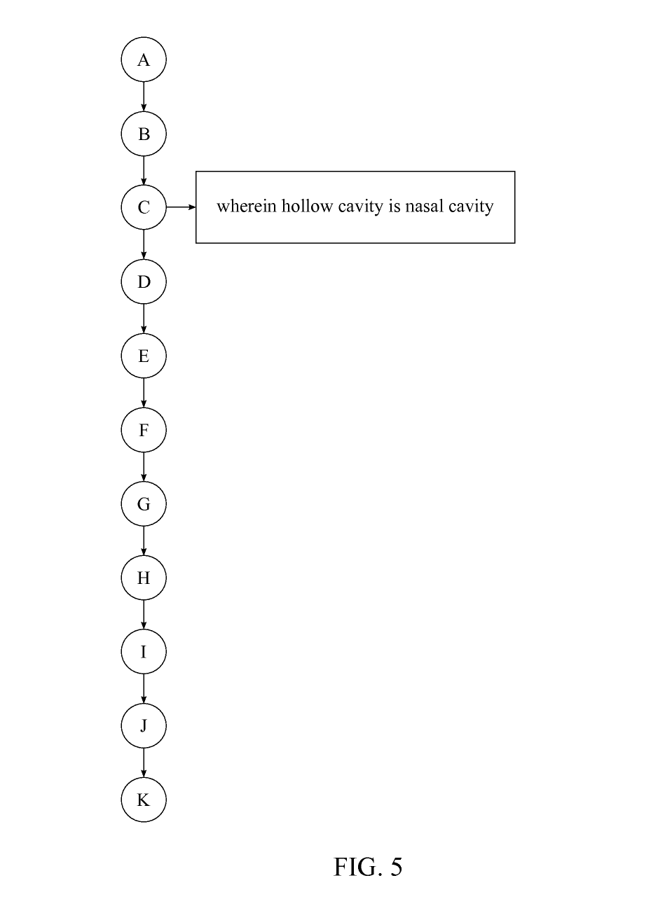

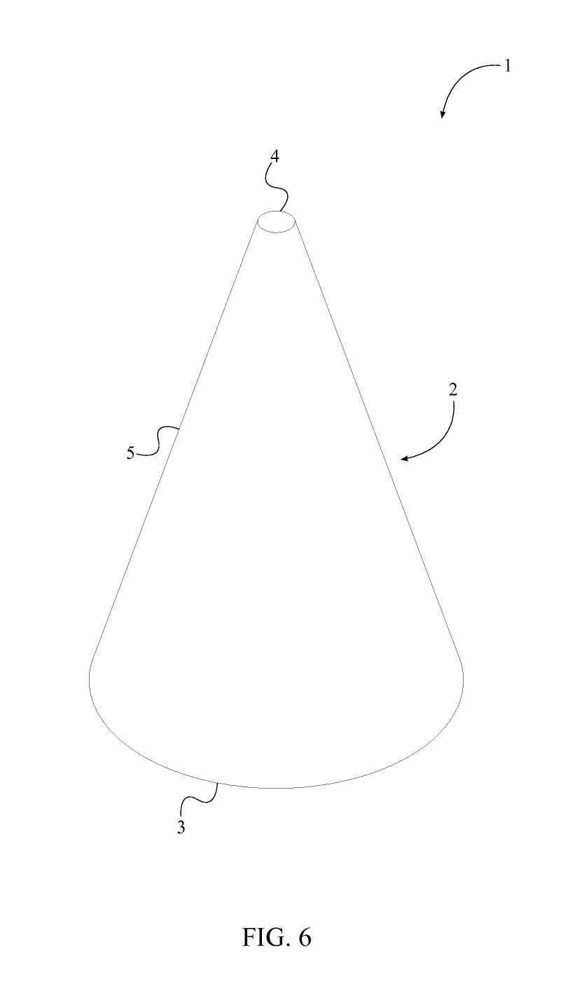

The method of cleaning and drying as introduced in the present invention is executed with a cleaning apparatus 1. The cleaning apparatus 1 is made of a flexible material in order to avoid causing any damage to the interior walls of an ear canal or the inner walls of a nasal cavity. The flexible material allows the cleaning apparatus 1 to deform according to the body part the cleaning apparatus 1 is used in. As seen in FIG. 6, a first structural body 2 of the cleaning apparatus 1 comprises a first wide base 3, a first narrow base 4, and a first conical lateral surface 5. The positioning of the first wide base 3, the first narrow base 4, and the first conical lateral surface 5 results in the first structural body 2 being cone shaped. In other words, the first conical lateral surface 5 tapers from the first wide base 3 towards the first narrow base 4. The cone shape of the first structural body 2 allows the user to maintain a firm grip on the first structural body 2. The cleaning apparatus 1 is intended to be used on a hollow cavity 12 on an external body surface. As shown in FIG. 4, the ear canal is considered as the hollow cavity 12. In another instance, the nasal cavity is considered to be the hollow cavity 12 as shown in FIG. 5. The size and shape of the hollow cavity 12 can vary from one user to another. However, since the cleaning apparatus 1 is made of a flexible, soft, and moisture absorbent material, the user can utilize the cleaning apparatus 1 accordingly. In addition to using the cleaning apparatus 1 in the hollow cavity 12, the cleaning apparatus 1 can also be used to apply or remove cosmetics.

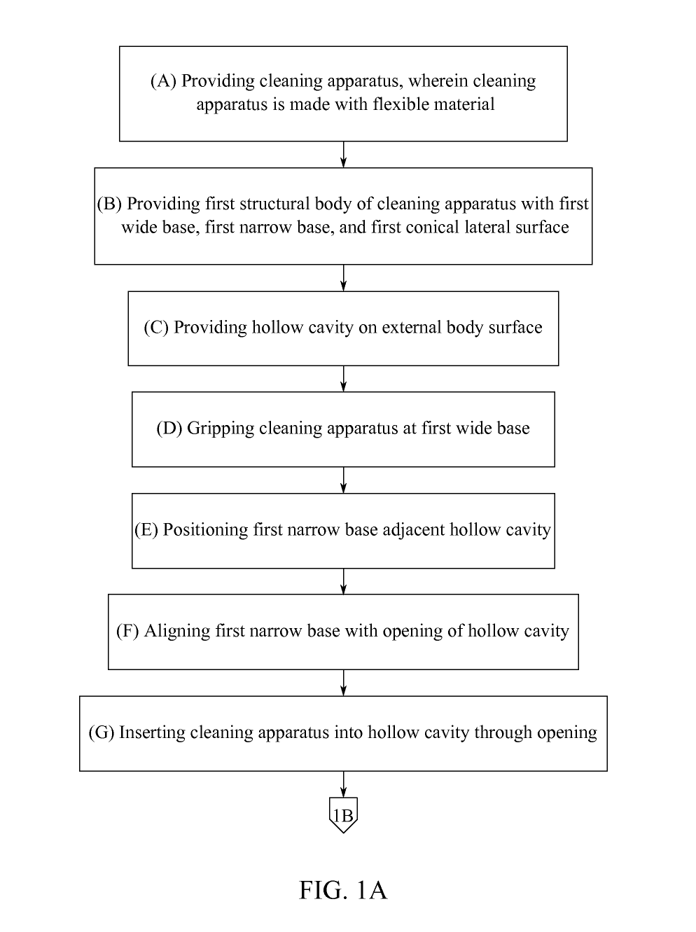

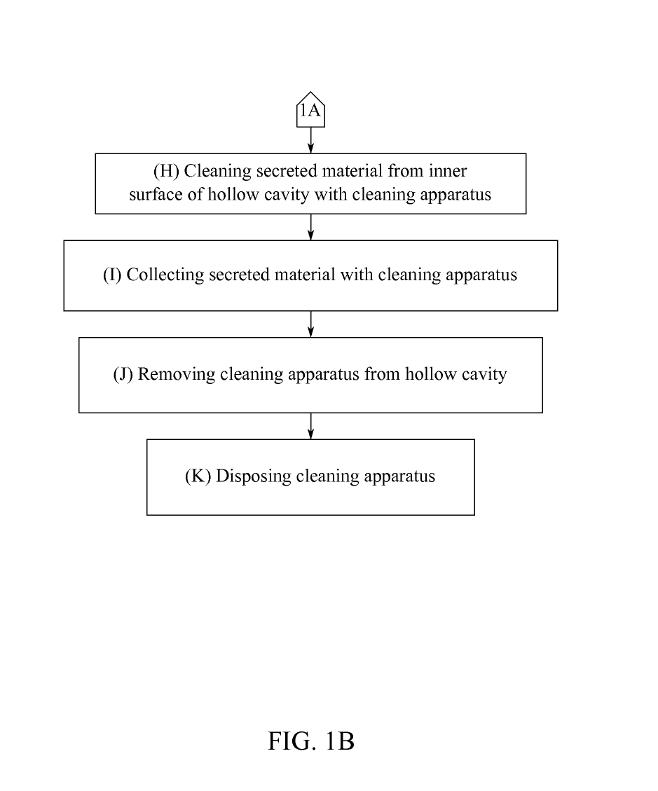

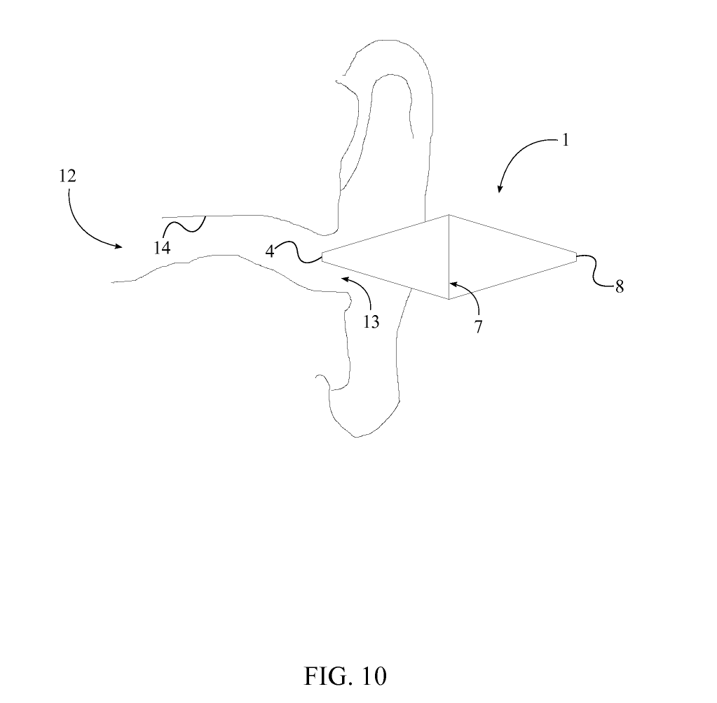

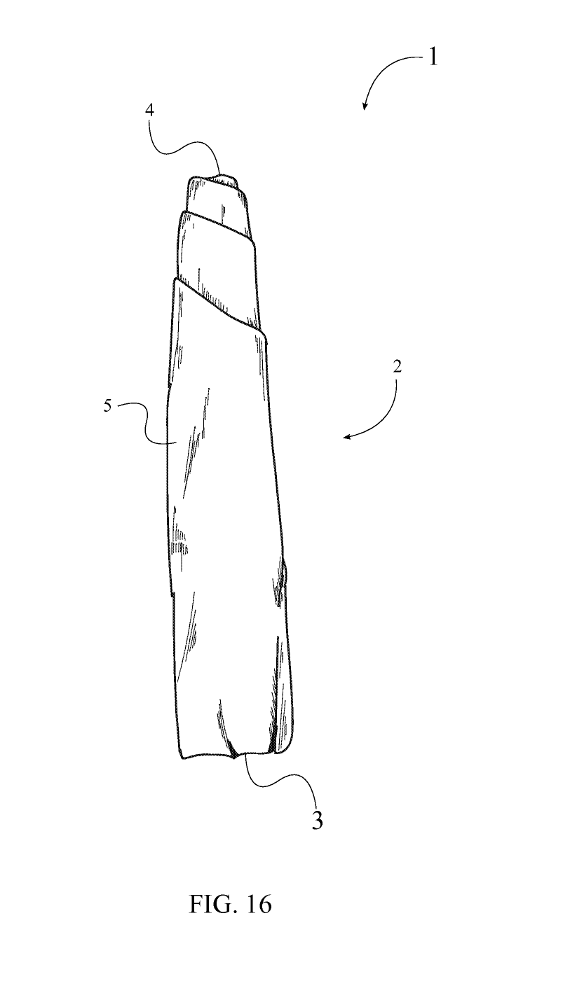

As illustrated in FIG. 1A and FIG. 1B, when executing the method introduced in the present invention, the cleaning apparatus 1 is gripped at the first wide base 3. The larger surface area of the first wide base 3 allows the user to establish a secure connection with the cleaning apparatus 1. Next, the first narrow base 4 is positioned adjacent the hollow cavity 12. In doing so, the first narrow base 4 is aligned with an opening 13 of the hollow cavity 12. If the hollow cavity 12 is the ear canal, the narrow base is positioned adjacent the opening 13 of the ear canal Likewise, if the hollow cavity 12 is the nasal cavity, the first narrow base 4 is positioned adjacent the opening 13 of the nasal cavity. When aligning the first narrow base 4 is complete as shown in FIG. 10, the cleaning apparatus 1 is inserted into the hollow cavity 12 through the opening 13. The first narrow base 4 is sufficiently sized in order to be comfortably inserted into the hollow cavity 12. As mentioned before, the cleaning apparatus 1 is made of a soft flexible which is also a moisture absorbent material. Therefore, when the cleaning apparatus 1 is within the hollow cavity 12, the first structural body 2 deforms to conform to an inner surface 14 of the hollow cavity 12 such that the cleaning apparatus 1 can be conveniently guided within the hollow cavity 12. The cleaning apparatus 1 is then guided as necessary to clean secreted material from the inner surface 14 of the hollow cavity 12. For added comfort, the first narrow base 4 can have additional padding compared to the first wide base 3. If the hollow cavity 12 is the ear canal, the cleaning apparatus 1 is utilized to clean and remove excess ear wax from the ear canal. On the other hand, if the hollow cavity 12 is the nasal cavity, the cleaning apparatus 1 is used to clean the mucus from the nasal cavity. When cleaning is complete, the secreted material is collected with the cleaning apparatus and the cleaning apparatus 1 is removed from the hollow cavity 12. In order to do so, the cleaning apparatus 1 is drawn out in a direction opposite to the previous direction in which the cleaning apparatus 1 was inserted in. When the cleaning process is complete and the cleaning apparatus 1 is removed from the hollow cavity 12, the cleaning apparatus 1 is disposed. In order to do so, the cleaning apparatus 1 is produced with a disposable material which is also biodegradable. As an example, the cleaning apparatus 1 can be built with a material which has material properties similar to tissue paper. FIG. 16 is an illustration of the cleaning apparatus 1, wherein the cleaning apparatus 1 is constructed by rolling tissue paper. Even though the tissue paper is rolled as seen in FIG. 16, any other comparable method can also be utilized to construct the cleaning apparatus 1.

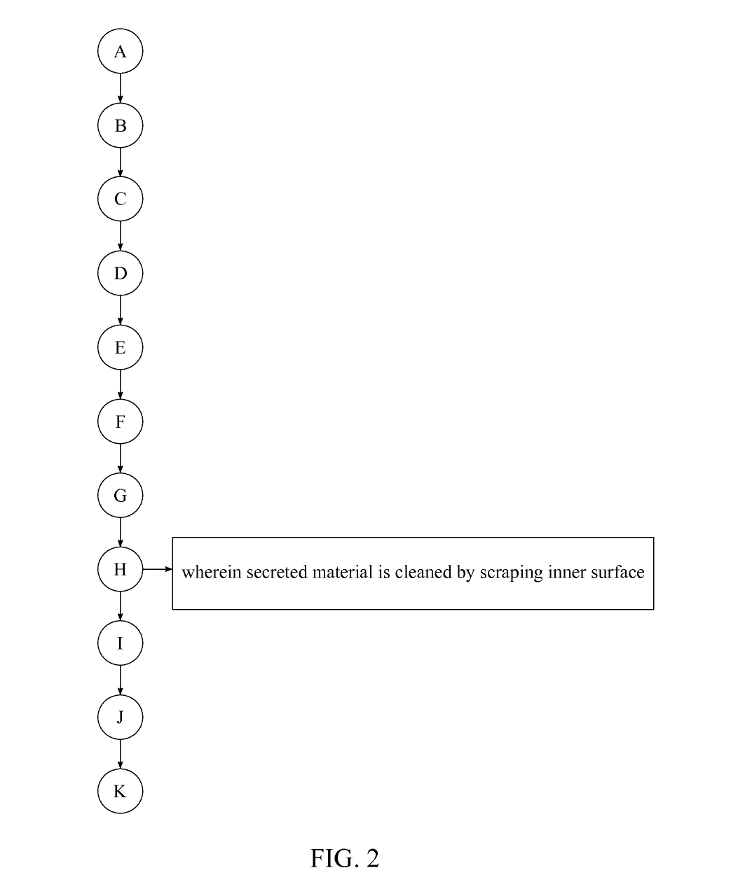

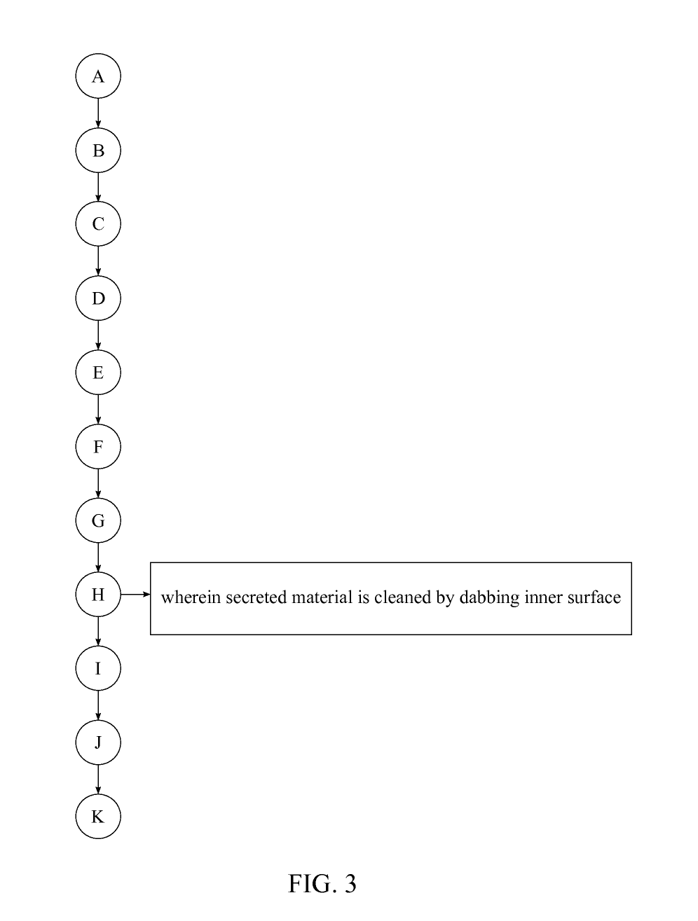

As shown in FIG. 2 and FIG. 3, the cleaning process can be completed through multiple methods. As an example, the secreted material can be cleaned by scraping the inner surface 14. The flexible material of the cleaning apparatus 1 and the softness of the cleaning apparatus 1 allows the user to safely scrape the inner surface 14 without damaging the inner surface 14. The cleaning process can also be completed by dabbing the inner surface 14. Once again, the material properties of the cleaning apparatus 1 allows the user to safely dab the inner surface 14. As an example, if the cleaning apparatus 1 is used within the ear canal, the cleaning apparatus 1 avoids damaging the ear drum.

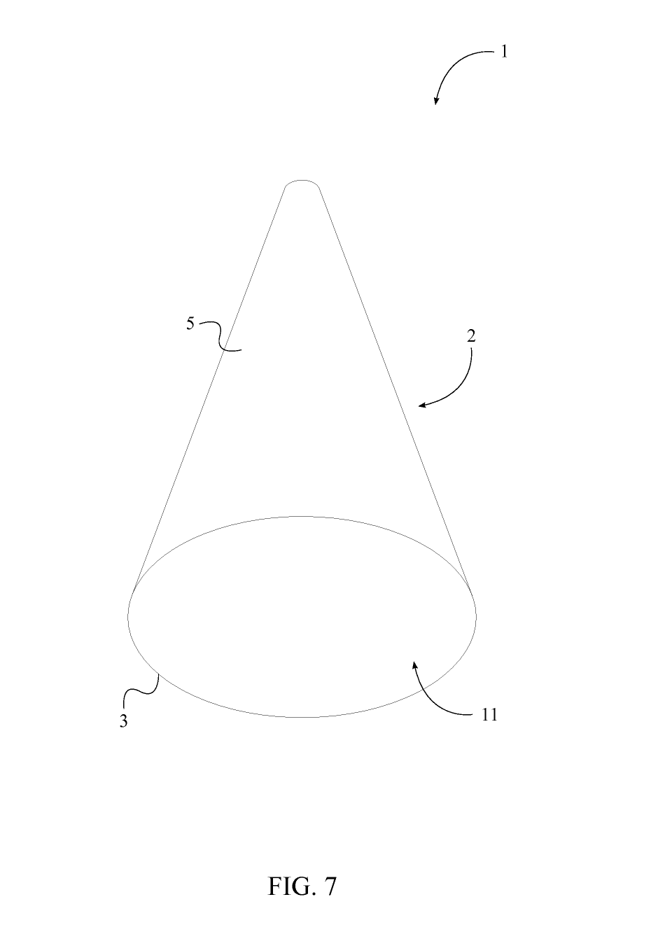

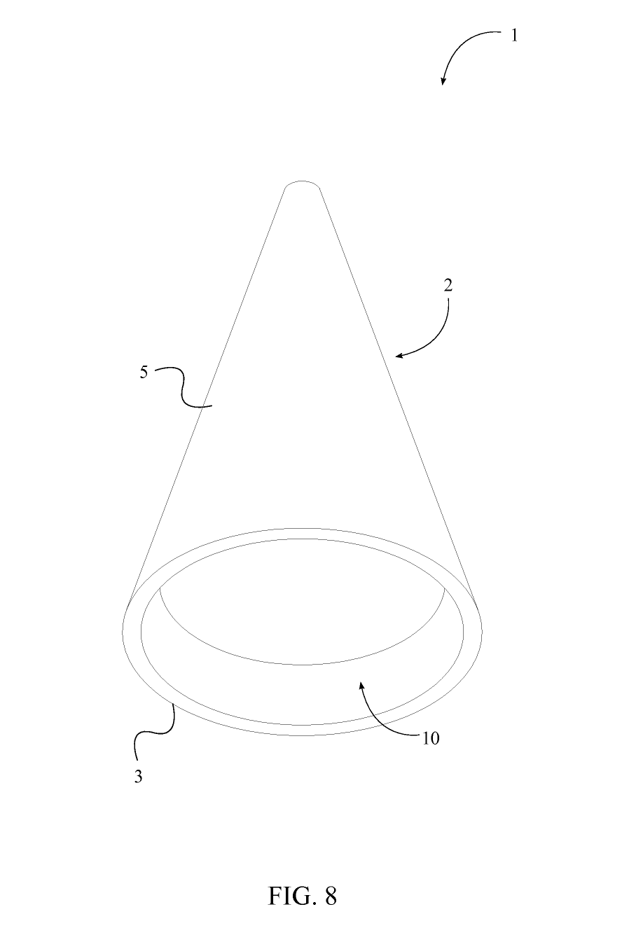

The cleaning apparatus 1 can be structured differently without hindering the overall functionality. As an example, the cleaning apparatus 1 can have a hollow core area 10 as illustrated in FIG. 8. In such instances, the hollow core area 10 is enclosed by the first wide base 3, the first narrow base 4, and the first conical lateral surface 5. The hollow core area 10 is beneficial in terms of reducing manufacturing costs and making the cleaning apparatus 1 lightweight. However, the cleaning apparatus 1 can also have a solid core area 11 as seen in FIG. 7. In such instances, the solid core area 11 is similarly enclosed by the first wide base 3, the first narrow base 4, and the first conical lateral surface 5. The solid core area 11 is beneficial in terms of providing the user with a flexible yet rigid structural body.

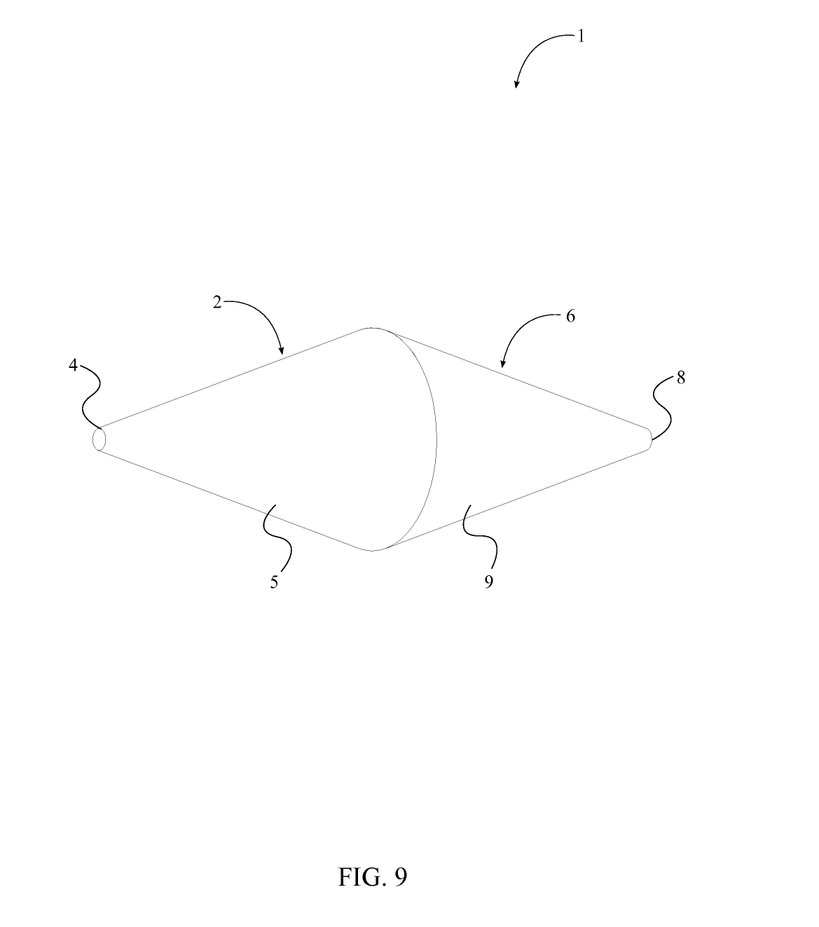

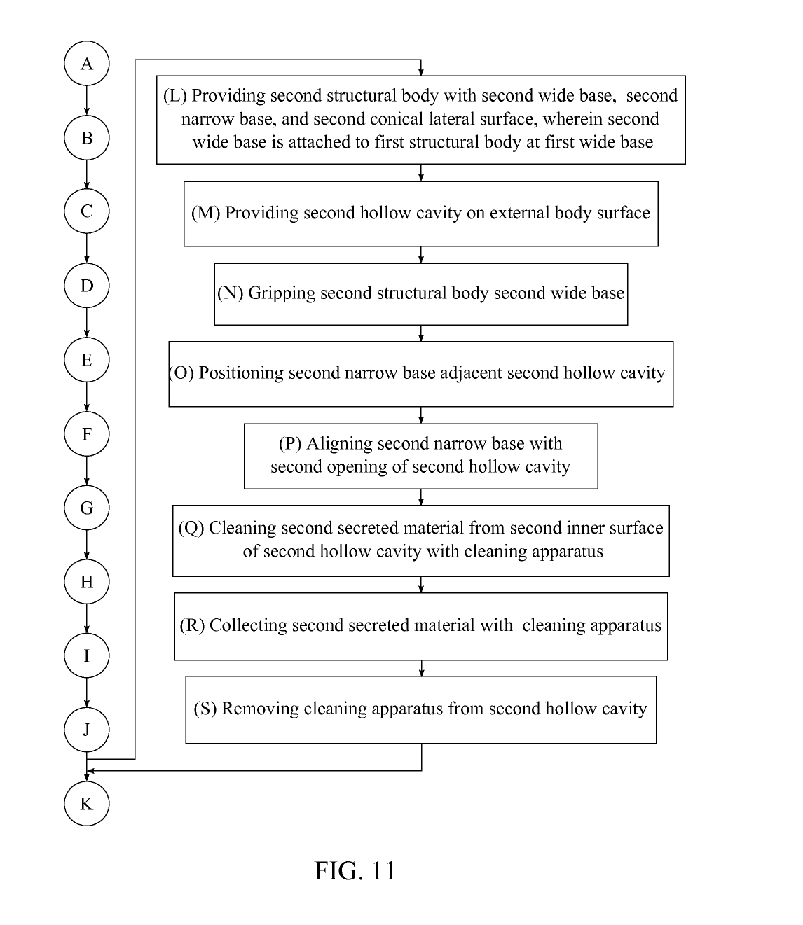

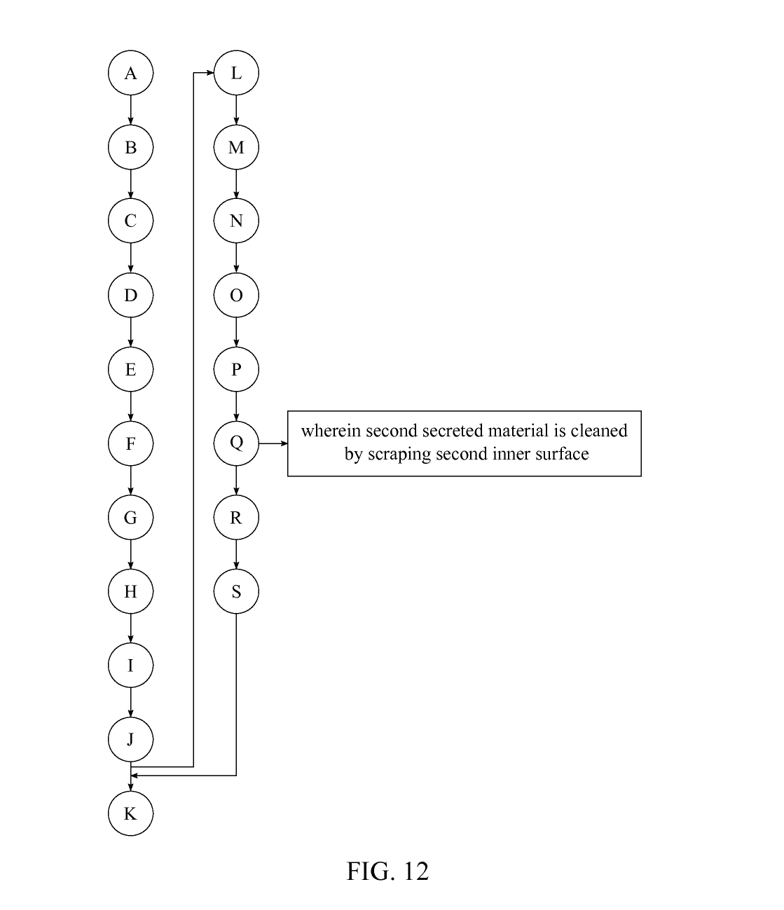

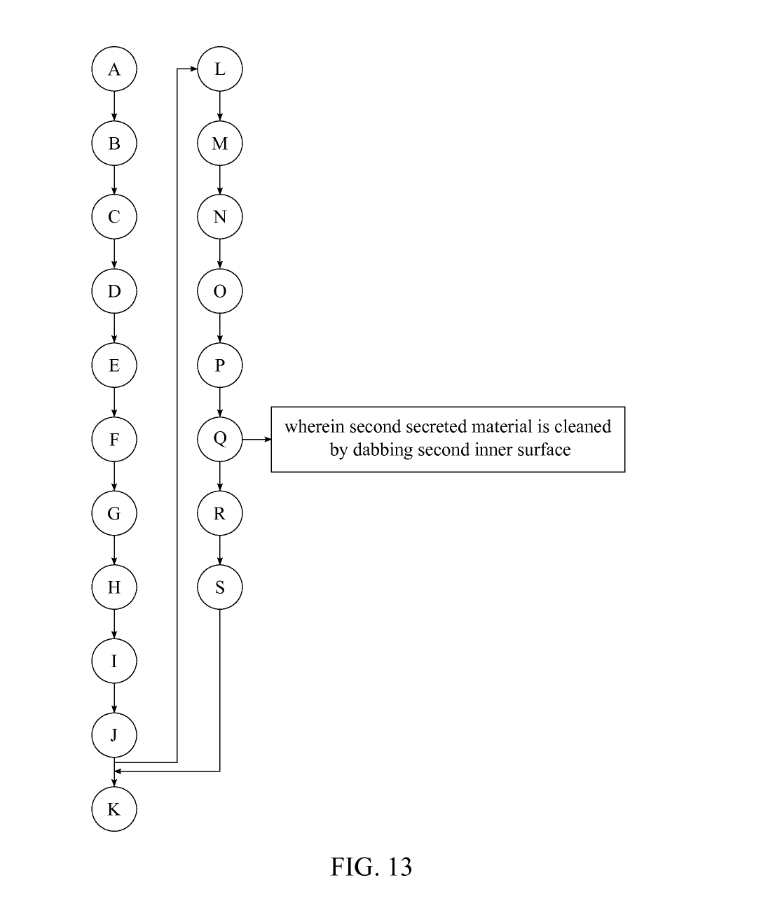

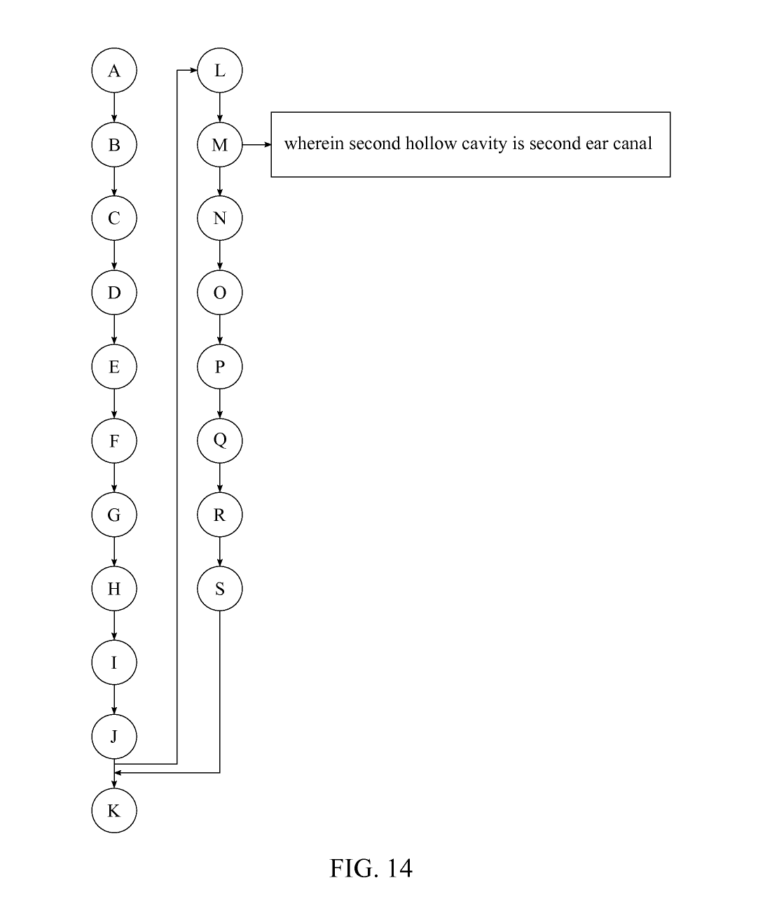

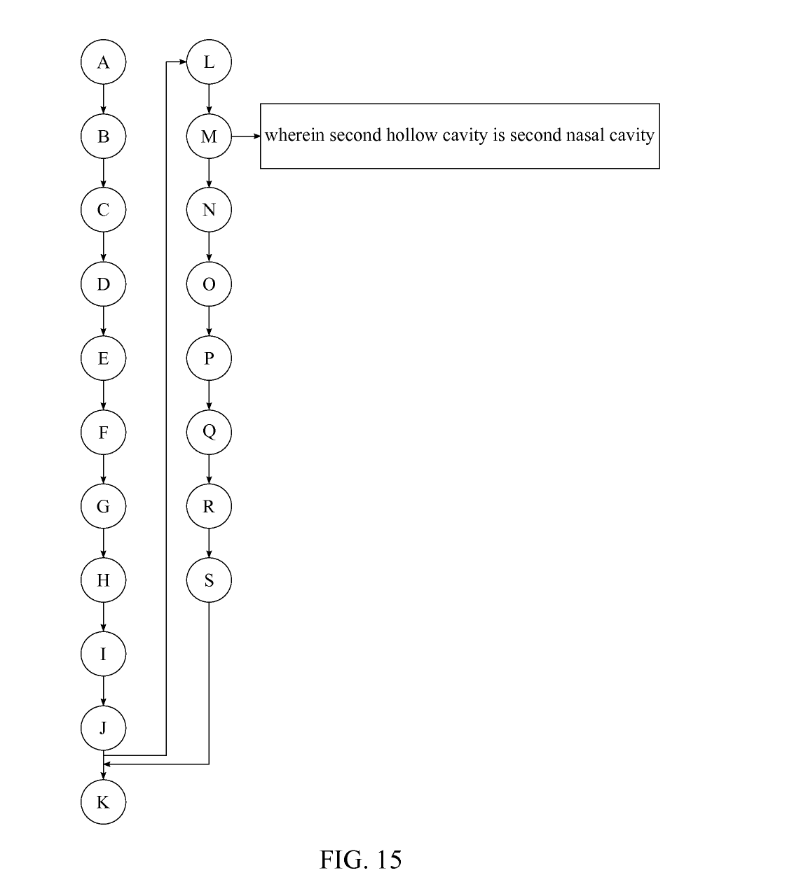

As discussed earlier, the cleaning apparatus 1 can have multiple configurations. As shown in FIG. 11, a second structural body 6 of the cleaning apparatus 1 can be designed with a second wide base 7, a second narrow base 8, and a second conical lateral surface 9. The second conical lateral surface 9 is tapered from the second wide base 7 towards the second narrow base 8 such that the second structural body 6 is also cone shaped. When the cleaning apparatus 1 includes the second structural body 6, the second wide base 7 is attached to the first structural body 2 at the first wide base 3. In other words, the first structural body 2 and the second structural body 6 are positioned facing opposite directions as illustrated in FIG. 9. The second structural body 6, when used with the first structural body 2 allows the user to utilize the cleaning apparatus 1 for multiple uses. Similar to using the first structural body 2 with the hollow cavity 12, the second structural body 6 is used along with a second hollow cavity of an external body surface. In doing so, the second structural body 6 is gripped at the second wide base 7 and the second narrow base 8 is positioned adjacent the second hollow cavity. As seen in FIG. 14 and FIG. 15, the second hollow cavity can be either a second ear canal or a second nasal cavity. As an example, if the first narrow base 4 was utilized on the left ear, the second narrow base 8 can be utilized on the right ear, wherein the right ear canal is considered the second hollow cavity. For user comfort, the second narrow base 8 can also be designed with additional padding. The second narrow base 8 is positioned such that the second narrow base 8 is aligned with a second opening of the second hollow cavity. Subsequently, the user proceeds to clean second secreted material from a second inner surface of the second hollow cavity with the cleaning apparatus 1. As shown in FIG. 12 and FIG. 13, the cleaning process can be completed by either scraping the second inner surface or by dabbing the second inner surface. The second inner surface is not damaged by the cleaning apparatus 1 since the second structural body 6 is also made of a soft and flexible material. When the cleaning apparatus is used to collect the second secreted material, the cleaning apparatus 1 is removed from the second hollow cavity. As a final step, the cleaning apparatus 1 is disposed accordingly.

Similar to the first structural body 2, the second structural body 6 can also be designed differently. In other words, the second structural body 6 can have the hollow core area 10 or the solid core area 11 according to user preference. As mentioned earlier, the hollow core area 10 can be used to minimize manufacturing costs. When the hollow core area 10 is utilized, the second wide base 7, the second narrow base 8, and the second conical lateral surface 9 encloses the hollow core area 10. In another instance, the solid core area 11 is enclosed by the second wide base 7, the second narrow base 8, and the second conical lateral surface 9. The solid core area 11 can be beneficial when the second structural body 6 requires rigidness.

By utilizing the method introduced by the present invention in the ear canal, the user can remove excess water and ear wax without damaging the inner surface 14 of the ear canal. Resultantly, the probability of having ear infections can be subsequently lowered or prevented. When the method introduced by the present invention is executed with the nasal cavity, mucus can be conveniently removed from the nasal cavity. As a result, viral infections and other serious medical conditions that occur due to bacteria is prevented. Additionally, the cleaning apparatus 1 can also be used for nose bleeds since the cleaning apparatus 1 is made of a moisture absorbent material. In doing so, the cleaning apparatus 1 is held at the first wide base 3 or the second wide base 7 such that the first narrow base 4 or the second narrow base 8 can be positioned within the nasal cavity. The soft and smooth material also allows the cleaning apparatus 1 to be used for applying or removing cosmetics. As an example, if the cleaning apparatus 1 is used to apply cosmetics, the cleaning apparatus 1 is initially dabbed in a preferred cosmetic material. The moisture absorbent nature of the material used for the cleaning apparatus 1 allows the user to conveniently transfer cosmetics onto the first structural body 2 or the second structural body 6. Next, the user utilizes the first narrow base 4 or the second narrow base 8 to apply the cosmetics in a preferred area. The smoothness of the material used for the cleaning apparatus 1 allows the user to apply cosmetics with improved convenience. If the user prefers to remove cosmetics using the cleaning apparatus 1, a preferred area of the skin is scraped or dabbed by holding the cleaning apparatus 1 at the first wide base 3 or the second wide base 7. The soft and smooth material of the cleaning apparatus 1 which is also moisture absorbent allows the user to remove cosmetics from the skin within a short time period.

Although the invention has been explained in relation to its preferred embodiment, it is to be understood that many other possible modifications and variations can be made without departing from the spirit and scope of the invention as hereinafter claimed.

What is claimed is:

1. A method for using an absorbent device for cleaning and drying purposes comprises the steps of:
providing a cleaning apparatus, wherein the cleaning apparatus is made with a flexible material;
providing a first structural body of the cleaning apparatus with a first wide base, a first narrow base, and a first conical lateral surface;
providing a hollow cavity on an external body surface;
gripping the cleaning apparatus at the first wide base;
positioning the first narrow base adjacent the hollow cavity;
aligning the first narrow base with an opening of the hollow cavity;
inserting the cleaning apparatus into the hollow cavity through the opening;
cleaning secreted material from an inner surface of the hollow cavity with the cleaning apparatus;
collecting the secreted material with the cleaning apparatus;
removing the cleaning apparatus from the hollow cavity; and
disposing the cleaning apparatus.

2. The method for using an absorbent device for cleaning and drying purposes as claimed in claim 1, wherein the secreted material is cleaned by scraping the inner surface.

3. The method for using an absorbent device for cleaning and drying purposes as claimed in claim 1, wherein the secreted material is cleaned by dabbing the inner surface.

4. The method for using an absorbent device for cleaning and drying purposes as claimed in claim 1, wherein a hollow core area is enclosed by the first wide base, the first narrow base, and the first conical lateral surface.

5. The method for using an absorbent device for cleaning and drying purposes as claimed in claim 1, wherein a solid core area is enclosed by the first wide base, the first narrow base, and the first conical lateral surface.

6. The method for using an absorbent device for cleaning and drying purposes as claimed in claim 1, wherein a moisture absorbent material is provided for the cleaning apparatus.

7. The method for using an absorbent device for cleaning and drying purposes as claimed in claim 1, wherein a disposable material is provided for the cleaning apparatus.

8. The method for using an absorbent device for cleaning and drying purposes as claimed in claim 1, wherein the hollow cavity is an ear canal.

9. The method for using an absorbent device for cleaning and drying purposes as claimed in claim 1, wherein the hollow cavity is a nasal cavity.

10. The method for using an absorbent device for cleaning and drying purposes as claimed in claim 1 further comprises the steps of:
providing a second structural body with a second wide base, a second narrow base, and a second conical lateral surface, wherein the second wide base is attached to the first structural body at the first wide base;
providing a second hollow cavity on an external body surface;
gripping the second structural body at the second wide base;
positioning the second narrow base adjacent the second hollow cavity;
aligning the second narrow base with a second opening of the second hollow cavity;
cleaning second secreted material from a second inner surface of the second hollow cavity with the cleaning apparatus;
collecting the second secreted material with the cleaning apparatus; and
removing the cleaning apparatus from the second hollow cavity.

11. The method for using an absorbent device for cleaning and drying purposes as claimed in claim 10, wherein the second secreted material is cleaned by scraping the second inner surface.

12. The method for using an absorbent device for cleaning and drying purposes as claimed in claim 10, wherein the second secreted material is cleaned by dabbing the second inner surface.

13. The method for using an absorbent device for cleaning and drying purposes as claimed in claim 10, wherein a hollow core area is enclosed by the second wide base, the second narrow base, and the second conical lateral surface.

14. The method for using an absorbent device for cleaning and drying purposes as claimed in claim 10, wherein a solid core area is enclosed by the second wide base, the second narrow base, and the second conical lateral surface.

15. The method for using an absorbent device for cleaning and drying purposes as claimed in claim 10, wherein the second hollow cavity is a second ear canal.

16. The method for using an absorbent device for cleaning and drying purposes as claimed in claim 10, wherein the second hollow cavity is a second nasal cavity.
